(19) United States
(12) Patent Application Publication
(10) Pub. No.: US 2024/0337669 A1
(43) Pub. Date: Oct. 10, 2024

(54) REPORTER LABELED ANNULAR CLOT SYSTEM FOR DIAGNOSIS AND RESEARCH

(71) Applicant: Indiana University Research and Technology Corporation, Bloomington, IN (US)

(72) Inventors: Nathan Joseph Alves, Zionsville, IN (US); Ziqian Zeng, Indianapolis, IN (US)

(21) Appl. No.: 18/681,772

(22) PCT Filed: Aug. 4, 2022
(86) PCT No.: PCT/US2022/039396

§ 371 Date: Feb. 6, 2024

Related U.S. Application Data
(60) Provisional application No. 63/230,286, filed on Aug. 6, 2021.

(51) Int. Cl. G01N 33/86; C07K 14/75; C12Q 1/37; G01N 33/96

(52) U.S. Cl. CPC G01N 33/86; C07K 14/75; C12Q 1/37; G01N 33/96; G01N 2333/75; G01N 2333/9726; G01N 2500/04

(57) ABSTRACT

Disclosed are compositions and methods of a microplate-based assay related to the field of thrombosis diagnosis, therapeutic screening, and drug development platforms.

### CROSS-REFERENCE TO RELATED APPLICATIONS

This application is a U.S. National Phase Application based on International Application No. PCT/US2022/039396 (published as WO 2023/014866), filed Aug. 4, 2022, which claims priority to U.S. Application Ser. No. 63/230,286, filed on Aug. 6, 2021, the disclosures of which are hereby incorporated by reference in their entireties.

### BACKGROUND OF THE DISCLOSURE

The present invention is generally directed to methods and compositions of a microplate-based assay related to the field of thrombosis diagnosis, therapeutic screening, and drug development platform.

Impaired fibrinolysis has long been considered to be a risk factor for venous thromboembolism (VTE). In response to blood vessel injury, human body initiates a hemostasis process, which is an innate series of actions followed by the clot formation. Fibrin is a protein that is primarily responsible for clotting and degrades through fibrinolysis at the end of hemostasis. A typical fibrinolysis process involves the activation of plasminogen by tissue plasminogen activator (tPA) to produce plasmin, an enzyme that digests fibrin. The kringle domains of plasmin bind to C (carboxy)-terminal lysine residues on fibrin to facilitate local clot digestion through its catalytic domain. The fibrinolytic system is also carefully regulated by inhibitors including type 1 plasminogen activator inhibitor (PAI-1) for tPA, α2-antiplasmin and α2-macroglobulin for plasmin. In acute thrombosis patients, thrombolytic drugs like recombinant plasminogen activators are often prescribed to accelerate plasmin conversion and clot digestion to offer a fast relief from life-threatening conditions.

Numerous assays have been developed to meet this challenge but all exhibit a variety of benefits and drawbacks limiting their overall utility. Chromogenic or antigenic assays are frequently used to measure individual clotting factor levels or activities. These assays, however; do not directly yield clinically representative results as they are unable to assess processes that involve native fibrinolytic events such as protein adsorption, multi-factor interactions, or plasmin-fibrin binding. Assays such as euglobulin clot lysis time (ECLT) and dilute whole-blood clot lysis time (DWCLT), have been extensively used since the 1980s. While these assays more globally represent in vivo clotting conditions they are inherently difficult to measure an accurate plasma fibrinolytic activity as they either test only a small sub-fraction of plasma or ignore the presence of varying amounts of fibrinogen in the test system. More recently, a plasma clot lysis time assay was developed by tracking a clot turbidity which is an optical measure of bulk clot structure. It has rapidly gained popularity for use in clinical studies due to its ease of implementation. Using tissue factor and tPA-treated patient plasma, a change in clot turbidity is monitored throughout the clot formation and lysis processes. However, this assay utilizes the patient's own plasma and results are often difficult to compare across samples due to variations from patient to patient. For example, clot lysis time is influenced by fibrinogen levels due to its impact on fibrin clot formation density and fiber thickness making interpretation of therapeutic effect by turbidity difficult. More importantly, the turbidity reading lacks microstructural or molecular interpretation capabilities. Although a fibrin fiber mass-length ratio can be extracted from turbidity measurements, the calculation relies on a number of assumptions that are sometimes difficult to determine. None of these assays allows for a physiologically relevant clot lysis determination that provides results than can directly be compared across patients in the presence and absence of a variety of therapeutic interventions.

Additional assays utilize exogenous fibrin as the substrate to assess fibrinolytic activity with the ability to offer physiologically relevant microstructures including binding moieties, cleavage sites and clot digestion depths. The fibrin plate method measures fibrinolytic potential by quantifying the lysed area of preformed fibrin in a petri-dish following incubation with a drop of patient plasma to its center. The assay is difficult to multiplex and quantification methods require standardization to allow for comparison across groups. Radioactively or fluorescently labeled fibrin clot lysis assays are historically developed to measure plasmin activity or plasma fibrinolytic potential. These assays incorporate molecular reporters such as ¹²⁵I or fluorescein isothiocyanate (FITC) labels in preformed fibrin clots and can be adapted to dynamically read clot digestion activity. Fibrinolysis is monitored by tracking the reporter signal released into the clot supernatant during digestion. The monitored fibrinolytic activity is independent of fibrinogen concentration in patient samples. Nonetheless, a common setup of such assays requires a frequent transfer of clot supernatant to a clean well for signal acquisition. This procedure largely interferes with the ongoing fibrinolytic reaction, introducing artifacts and making it difficult for multiplexing, standardization and utilization under fast-responding clinical environments. More importantly, both isotopic iodine and FITC molecules conjugate fibrinogen non-specifically at primary amines of lysine residues. These conjugations are irreversible and could dramatically affect fibrin polymerization process resulting in impaired fibrin structures. When conjugations block C-terminal lysine residuals or potential fibrin digestion sites on fibrin, fibrinolysis becomes less representative as a constant exposure of these residues is critical. Therefore, results of these assays may not have good clinical relevance unless a characterization of reporter-labeled fibrin clots is thoroughly addressed.

Although efforts have been made over the years, no existing fibrinolytic assessment assays appears to genuinely aid in thrombosis diagnosis or offer a reliable testing platform for therapeutic development. Despite the fast development of novel drug agents, the clinical translation rate is not promising for the lack of representative drug testing platform. Therefore, due to the intricacy of the fibrinolytic system, an ideal fibrinolytic platform assay for diagnosis and drug testing should use a standardized substrate and be capable of dynamically monitoring the entire fibrinolytic process at a physiologically relevant scale and offer reproducible and comparable results. Accordingly, there exists a need for fibrinolytic assessment assays for thrombosis diagnosis, therapeutic screening, and drug testing.

### BRIEF DESCRIPTION OF THE DISCLOSURE

The present disclosure is generally related to methods and compositions of a microplate-based assay related to the field of thrombosis diagnosis, therapeutic screening, and drug development platform.

In one aspect, the present disclosure is directed to a labeled fibrin clot comprising: unlabeled fibrinogen and labeled fibrinogen at a ratio of unlabeled fibrinogen:labeled fibrinogen ranging from 0:1 to about 1:0, wherein the labeled fibrinogen comprises from 1 to 34-labels per fibrinogen.

In one aspect, the present disclosure is directed to a method of analyzing fibrin clot lysis, the method comprising: contacting a thrombolytic agent with a labeled fibrin clot, wherein the labeled fibrin clot comprises a ratio of unlabeled fibrinogen:labeled fibrinogen ranging from 0:1 to about 50:1, wherein the labeled fibrinogen comprises from 1 to 34-labels per fibrinogen, and a clot-free detection path; and analyzing the labeled fibrin clot.

In one aspect, the present disclosure is directed to a method of determining a patient's response to a thrombolytic therapy, the method comprising: obtaining a plasma sample from the patient; mixing the plasma sample with a labeled fibrinogen clotting solution, the labeled fibrinogen clotting solution comprising: unlabeled fibrinogen and labeled fibrinogen at a ratio of unlabeled fibrinogen:labeled fibrinogen ranging from 0:1 to about 1:0, wherein the labeled fibrinogen comprises from 1 to 34-labels per fibrinogen; forming a labeled fibrin clot by mixing the labeled fibrinogen clotting solution with a clotting activator; forming a clot-free detection path in the labeled fibrin clot; contacting the labeled fibrin clot with a thrombolytic therapy; and analyzing the labeled fibrin clot.

In one aspect, the present disclosure is directed to a high-throughput screening method for analyzing a candidate for blood clot digestion and thrombolytic agents, the method comprising: forming a labeled fibrin clot in a well, wherein the labeled fibrin clot is formed by: labeling fibrinogen to obtain a labeled fibrinogen, wherein labeled-fibrinogen comprises from 1 to 34-labels per fibrinogen; mixing the labeled fibrinogen with an unlabeled fibrinogen at a ratio ranging from about 0:1 to about 1:0 (labeled fibrinogen:unlabeled fibrinogen) to form a labeled fibrinogen clotting solution; contacting the labeled fibrinogen clotting solution with a clotting activator; placing the labeled fibrinogen clotting solution with the clotting activator in the well; performing a clotting step by incubating the labeled fibrinogen clotting solution with the clotting activator in the well for a sufficient time to allow the fibrinogen clotting solution with the clotting activator to form a labeled fibrin clot; and forming a clot-free detection path in the labeled fibrin clot; adding a candidate blood clot digestion or thrombolytic agent to the labeled fibrin clot; and analyzing the labeled fibrin clot.

In one aspect, the present disclosure is directed to a method for monitoring fibrinolytic potential of a clinical sample to assess a patient's thrombosis risk. The method comprises the formation of a fluorescently labeled annular fibrin clot; the addition of a clinical sample; and a real-time fluorescence monitoring for clot digestion; wherein the clinical sample solution is anticoagulated platelet-poor plasma, platelet-rich plasma, or whole blood with or without the addition of plasminogen activators (or other clot formation/clot reducing agents) including but not limited to tissue plasminogen activator (t-PA), urokinase-type plasminogen activator (u-PA), streptokinase plasminogen activator (strepto-PA), recombinant tissue plasminogen activator (rt-PA), recombinant plasminogen activator (r-PA), tenecteplase (TNKASE®), and wherein the used anticoagulant is citrate, Ethylenediaminetetraacetic acid (EDTA), or heparin. In one embodiment, a standard curve for fibrinolytic potential is established by plotting digestion rates across a dilution series of physiological levels of plasminogen/plasmin or normal patient blood samples. Thrombosis risk of a patient is determined by comparing patient assay results with the standard curve.

In another aspect, the present disclosure is directed to a method for guiding a personalized treatment regimen for acute thrombosis or bleeding. The method comprises the formation of a annular clot substrate; the addition of therapeutic intervened patient blood samples; and a real-time fluorescence monitoring for clot digestion; wherein the clot substrate is a fluorescently labeled fibrin clot at a physiological concentration or fluorescently labeled patient own clot formed using their platelet-poor plasma, platelet-rich plasma, or whole blood and wherein the therapeutic agents are plasminogen activators, plasmin and its derivatives, fibrinolytic inhibitors, antiplasmin, or PAI-1, including synthetic and recombinant forms of plasminogen activators, plasmin and its derivatives, fibrinolytic inhibitors, antiplasmin, or PAI-1. The most efficient therapeutic and its dosage can be determined by comparing fluorescence curve and overall digestion rates across arrays of wells containing different therapeutics. In this manner a patient sample can be screened for a variety of thrombolytic drugs or dosages to determine a personalized delivery of the drug limiting bleeding risk and eliminating the trial and error associated with administering multiple drugs over time that may or may not all be efficacious for each unique patient.

In another aspect, the present disclosure is directed to a method for assessing bleeding risk of a clinical sample. The method comprises the formation of an annular clot substrate; the addition of a thrombolytic agent; and a real-time fluorescence monitoring for clot digestion; wherein the annular clot substrate is a clot formed using the patient's own platelet-poor plasma, platelet-rich plasma, or whole blood with or without the mixing of fluorescently tagged fibrinogen; and wherein thrombolytic agents are plasminogen activators including but not limited to t-PA, u-PA, strepto-PA, rt-PA, r-PA, TNKase. In some embodiment, a standard fluorescence release rate is monitored using plasminogen activator intervened healthy patient blood samples. Patient bleeding risk is determined by comparing results with the standard fluorescence release rate.

In another aspect, the present disclosure is directed to a method for screening thrombolytic or fibrinolytic drugs (drug screening/testing/development platform). The method comprises the formation of a reproducible annular clot substrate; the addition of a drug sample; and a real-time fluorescence monitoring for clot digestion; wherein the drug sample is dissolved in buffer with plasminogen or plasma and wherein the annular clot substrate is a fluorescently labeled fibrin clot, plasma clot, whole blood clot, or clot analog and wherein fluorescence molecules are incorporated for tracking clot digestion. Drug efficacy is assessed by comparing fluorescence release curve and overall digestion rate across drugs or controls. In this manner the annular clot assay can be used to compare current therapeutic molecules to next generation clot digestion molecules against a reproducible, and representative, clot substrate. This has the added benefit of reducing the need for animal experimentation to ensure animal use in research and development are only utilized when absolutely necessary.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1A depicts the crystal structure of fibrinogen and the modification of lysine residues through a covalent fluorescence tagging.

FIG. 1B depicts one method to incorporate fluorescently tagged fibrinogen for fluorescently labeled fibrin formation. The fluorescently tagged fibrinogen are mixed with solutions that contain fibrinogen to make clotting mixtures.

FIG. 2 is an illustration depicting the default absorbance or fluorescence signal detection mechanism of a commercially available spectrometer or fluorometer.

FIG. 3A is a schematic illustrating an exemplary embodiment of a 3×2 mold insert for a well plate.

FIG. 3B is a side-view schematic illustrating steps in the formation of an annular clot using a mold insert.

FIG. 3C is a photograph of a 3D printed 3×2 insert for a 96-well microplate.

FIG. 3D are photographs depicting a top-view of a 3×2 annular clot and a bottom-view of a 3×2 annular clot.

FIGS. 4A-4C are representative tracing curves of turbidity assay (FIG. 4A) and Thromboelastography (TEG) assay (FIG. 4B) for varying 12FITC labeled human fibrinogen (12FhF) levels in 12FITC-fibrin clot. Turb^{Max} (FIG. 4C) and TEG^{Max} (FIG. 4D) (Maximum amplitude) of 3, 7 and 12-FITC-fibrin clots at different FhF levels were compared. Data were normalized by values of human fibrinogen control groups. * denotes significant differences (P value <0.05) between FhF groups and hFbg ctrl group.

FIGS. 5A-5C are representative SEM images and graphs depicting fiber properties of FITC labeled human fibrin formed by neat and unmodified fibrinogen. SEM images of FITC labeled human fibrin formed by 1 U/mL thrombin and 3 mg/mL neat and unmodified fibrinogen mixed 3,7,12-FITC-fibrinogen at (FIG. 5A) 4,000× and (FIG. 5B) 35,000×. Scale bars were shown as 5 μm and 500 nm, respectively. FIG. 5C are graphs depicting average fiber diameter (nm), average pore size (μm²) and total pore area % were reported as bar plots and data were compared with unmodified fibrin controls using *, ** and *** denoting p values <0.05, 0.001, and 0.0001.

FIGS. 6A-6D depict tagging consistency by confocal microscopy. Representative confocal microscope images (40× objective with 1.5× digital zoom-in, excitation energy at 1%) of clots formed by neat 3,7,12-FhF (FIG. 6A), and 3FhF (10:1), 7FhF (30:1) and 12FhF (50:1) (FIG. 6B). Fluorescence intensity distribution were shown for physiologically relevant FhF clots. Integrated density were shown for neat FhF (FIG. 6C) and physiologically relevant (FIG. 6D) clots in bar plots with brackets denoting pairs of groups that exhibit significant differences (P<0.05). Image brightness of physiologically relevant FhF clots were adjusted for structure visualization. Scale bar=20 μm.

FIGS. 7-9 are graphs depicting the stability of FITC tagged human fibrinogen and FITC labeled human fibrin over time and under different storage conditions. FIG. 7 depicts the stability of fluorescently labeled fibrin clot over 56 days by monitoring clot turbidity at 550 nm. FIG. 8 depicts the fluorescence (Ext. 495 nm, Em. 519 nm) stability of FITC tagged fibrinogen over 4 freeze-thaw cycles. FIG. 9 depicts the photobleaching rate of 50, 125, 250 μM 12FhF exposed to dynamic fluorescence reads inside a spectrometer. Data are shown as mean #standard deviation.

FIGS. 10A & 10B are graphs depicting tracing curves of fluorescence release of PR-12FhF at varying plasmin concentration and the derivation of FLU200 for PR-12FhF at 1.5 U/mL where FLU 200 is a measure of the time it takes to reach a release of 200 fluorescence unit (FIG. 10A). FLU200 for PR-12FhF over varying plasmin concentration (FIG. 10B). Brackets denoting pairs of groups that exhibit significant differences (P<0.05).

FIGS. 11A & 11B are graphs depicting fluorescence release rate (VFR) in neat and physiologically relevant (PR)-12FhF annular clot lysis assay where data were shown at the double-logarithmic scale. Analytical sensitivity determination curves of annular clot lysis assay by plotting data points below 0.05 U/mL plasmin. Human plasmin was used as an example and dose-response activity (0.001-1.5 U/mL) determined using 3 mg/mL physiologically relevant (fibrinogen: 12FITC-fibrinogen=50:1) and neat 12FITC-fibrin annular clot. Fluorescence release rate (VFR) plotted in a logarithmic scale (FIG. 11A) and in a normal scale at lower end of plasmin concentrations (FIG. 11B). Data shown as mean±standard deviation.

FIGS. 12A & 12B depict the examination of sample fibrinolytic potential or fibrinolytic drug efficacy by tracking absorbance (280 nm) using 0.1 U/mL plasmin as an example to digest different FITC labeled fibrin annular clots formed by 1:0 (−F), 1:5 (−R5), and 1:10 (−R10) ratios of 3, 7 and 12 FITC tagged fibrinogen to unmodified fibrinogen at 3 mg/mL (FIG. 12A). Digestion rate was determined and shown as mean±standard deviation (FIG. 12B).

FIG. 13 depicts the examination of sample fibrinolytic potential or thrombolytic drug efficacy by tracking the absorbance at 494 nm using 0.1 U/mL plasmin as an example to digest different FITC labeled fibrin annular clots formed by 3 mg/mL pure 3 (−3F), 7 (−7F), and 12 FITC (−12F) tagged fibrinogen.

FIGS. 14-16 depict the determination of sample fibrinolytic potential or thrombolytic drug efficacy in the presence of inhibitors by tracking fluorescence (Ext. 495 nm and Em. 519 nm) in 3 mg/mL physiologically relevant 12FITC-fibrin annular clots formed at 1 U/ml thrombin. 0.5 U/mL plasmin with plasmin inhibitors, such as pentamidine (5-250 μM) (FIGS. 14A & 14B), tranexamic acid (TXA) (0-1 M) (FIGS. 15A & 15B) and Aminocaproic acid (ACA, 0-3 M) (FIGS. 16A & 16B) used as examples. Representative tracing curves (left) are plotted, and digestion rates (right) are shown as mean±standard deviation.

FIGS. 17A-17F are graphs depicting digestion rates (primary axis, Abs/min or FLU/min) and plasmin activity (secondary axis, U/mL) by fixed tPA and varying PLG in S2251 assay (FIG. 17A), fixed tPA and varying PLG in PR-12FhF annular clot lysis assay (FIG. 17B), fixed PLG and varying tPA in S2251 assay (FIG. 17D) and fixed PLG and varying tPA in physiological relevant (PR)-12 FhF annular clot lysis assay (FIG. 6E). FLU200 by PR-12FhF were shown in bar plots at varying PLG (FIG. 17C) and varying tPA (FIG. 17F) experiments with brackets denoting pairs that have significant differences. Absorbance was monitored at 405 nm and fluorescence was tracking at Ext. 495 nm and Em. 519 nm. PR fibrin clots were formed using 3 mg/mL fibrinogen mixture (fibrinogen: 12FITC-fibrinogen=50:1). Plasmin activities were converted by plugging the digestion rate values in linear regression equations derived in FIG. 5. Plasmin activities were of two orders of magnitude faster in annular clot lysis assay compared to S2251 assay.

FIG. 18 is a graph depicting the determination of fibrinolytic potential (plasminogen activity) of swine venous plasma by the addition of 0, 50, 200, 500, 1000 ng/ml human tPA using physiologically relevant 12FITC-human fibrin annular clots. Swine blood was collected in 3.2% sodium citrate through a venipuncture at jugular vein. Digestion rates (VFR) were shown in a box plot with error bars depicting standard deviations.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Disclosed herein is a representative reporter labeled real-time clot lysis assay that uses a clot-free detection path. As used herein, “clot-free detection path” refers to an area or region in the labeled fibrin clot that is devoid of the clot material. The clot-free detection path provides a clear light path for absorbance and fluorescence excitation and emission by taking advantage of the restricted signal acquisition mechanism within a plate reader. A particularly suitable geometry of the clot used in the assays described herein is an annular shaped clot. Suitable alternatives to the annular clot-free detection path include star, square, oval, rectangle, triangle, and other shapes that allow for a clear light path. The labeled fibrin clot is a hydrogel-based structure and can be formed either in situ or externally and placed inside a well to function as a solid-phase substrate. Artificial clots are obtained by activating a fibrinogen-based clotting solution with clotting activators (also referred to herein as clot initiators) such as thrombin, recalcification, tissue factor, phospholipids, and combinations thereof followed by an incubation period for clots to solidify. Clot formation can further include platelet activation.

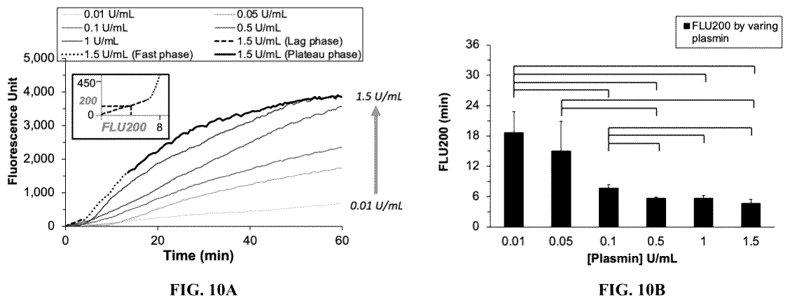

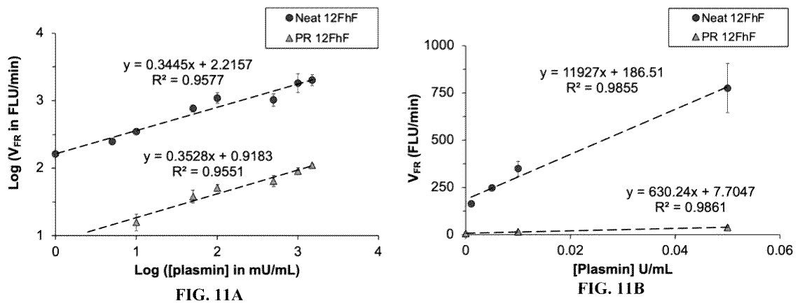

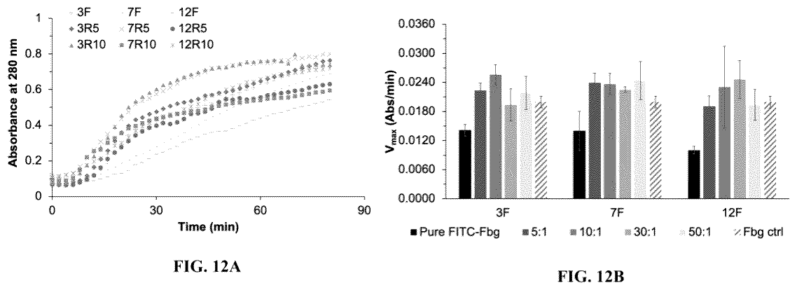

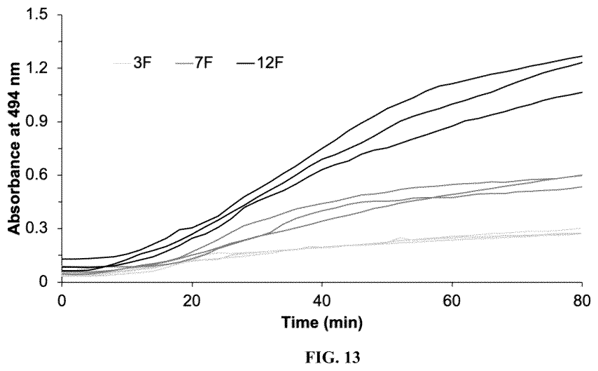

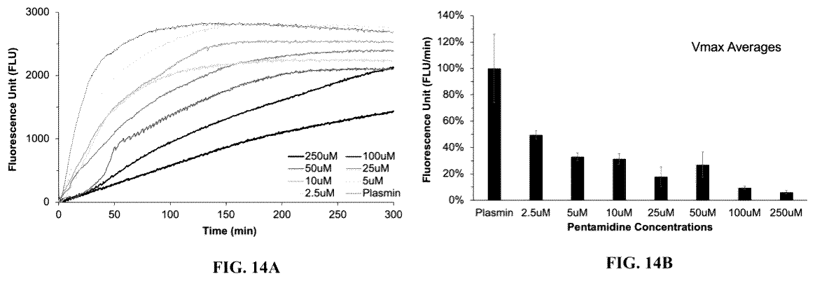

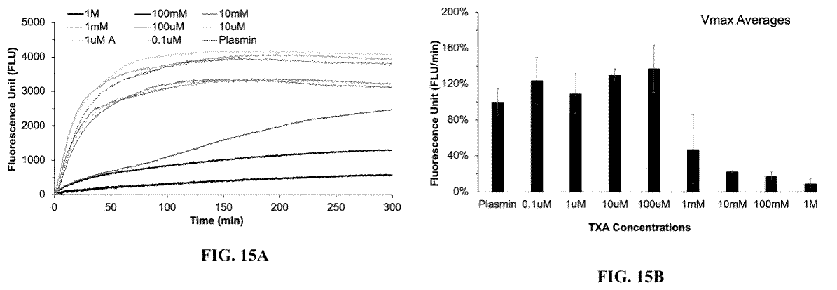

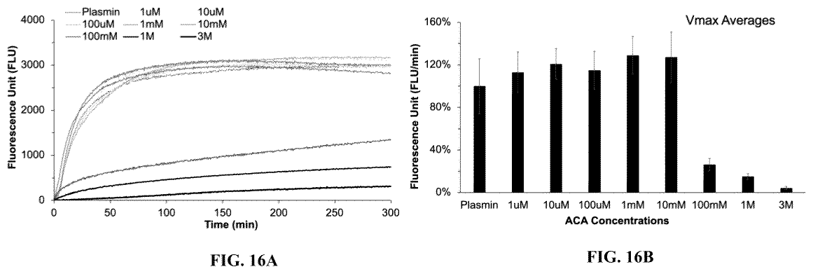

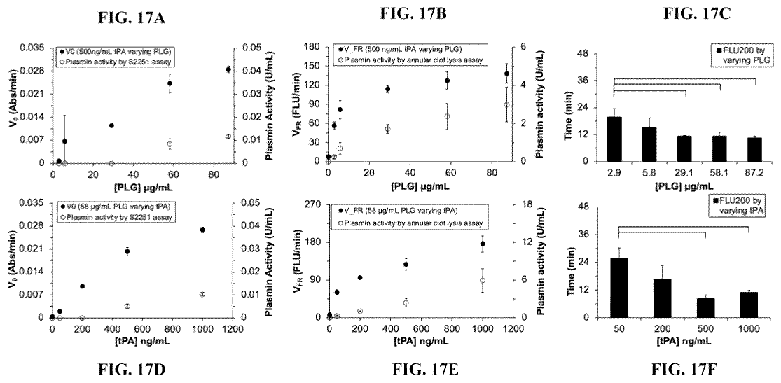

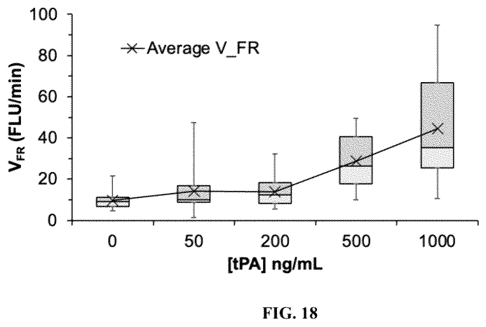

A representative real-time clot lysis assay present herein includes a fibrin clot substrate and sample solution that is placed in the well center, wherein a standard clot substrate is formed by initiating a mixture of reporter tagged fibrinogen and unmodified fibrinogen with thrombin. FIGS. 10-13 illustrate a plasmin example sample solution. FIG. 17 illustrates tissue plasminogen activator and plasminogen example sample solutions. FIG. 18 illustrates tissue plasminogen activator and pig venous blood example sample solutions.

In one embodiment, the clot-free detection path in the clot is formed during the clot formation. The clot-free detection path shape can be formed using molding inserts that match the desired shape of the clot-free detection path. Clot shaping molds (mold inserts) can be fabricated using 3D printing, injection molding, and extrusion. For example, a molding insert in the shape of a circle is inserted into the labeled fibrin clotting solution during a clot formation step. The molding insert is also fabricated to fit any well plate geometry or number (e.g., single well and multi-well plates). When using a molding insert, the clot-free detection path is formed in its final shape as the coagulation process is carried out with the molding insert in place (similar to a gelatin mold). After clotting, the molding insert is removed, leaving a clot-free space in the labeled fibrin clot. In another embodiment, the clot-free detection path in the clot is formed after clot formation. For example, following the clot formation step a shape sectioning tool or instrument such as a biopsy punch is used to remove a portion of the clot, leaving a clot-free space in the labeled fibrin clot.

Clotting conditions can be adjusted by varying conditions under which clotting takes place. These different conditions include varying temperature for clotting, varying calcium levels in the clotting solution, varying pH, varying ionic strength of blood products and/or buffer, and combinations thereof. The clotting conditions can also be adjusted by adding in agonists, such as adenosine diphosphate (ADP) and arachidonic acid to modulate platelet activity.

Representative clotting solutions include: a mixture of fluorescently tagged fibrinogen (alternatively tagged fibrinogen with any number of different reporter molecules) mixed with native fibrinogen, platelet-rich plasma, platelet-depleted plasma, whole blood, blood analogs that contain fibrinogen at any blending ratio, other clotting cascade proteins, synthetic molecules that induce or reduce clotting, and combinations thereof. The system can be utilized similarly regardless of species of origin (i.e., human, monkey, bovine, porcine, rat, mouse, rabbit, etc.).

As used herein, “a subject in need thereof” and “patient in need thereof” refers to a subject having, suspected of having, susceptible to, or at risk of a specified disease, disorder, or condition. More particularly, in the present disclosure the methods of treating thrombosis, methods for monitoring a subject's response to thrombolytic drugs, methods of determining a subjects bleeding risk, and methods of diagnosing thrombosis risk are to be used with a subset of subjects who are susceptible to or at elevated risk for experiencing thrombosis and bleeding. Subjects may have, be suspected of having, be susceptible to, or be at risk for thrombosis or bleeding risk due to family history, age, environment, and/or lifestyle.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions. As used herein, “susceptible” and “at risk” refer to having little resistance to a certain disease, disorder or condition, including being genetically predisposed, having a family history of, and/or having symptoms of the disease, disorder or condition.

In one aspect, the present disclosure is directed to labeled fibrin clot. The labeled fibrin clot includes unlabeled fibrinogen and labeled fibrinogen at a ratio of unlabeled fibrinogen:labeled fibrinogen ranging from 0:1 to about 1:0, wherein the labeled fibrinogen comprises from 1 to 34-labels per fibrinogen.

Fibrinogen is labeled via lysine residues, N-glycosylation sites, disulfide bonds, and combinations thereof. Lysine residue conjugates can include labeling from 1 to 16-labels per fibrinogen, including 1 to 13-labels, including 1 to 12-labels, including 3 to 13 labels, and including 3 to 12-labels. N-glycosylation site conjugates can include labeling from 1 to 8 per fibrinogen. Disulfide bond conjugates can include labeling at 1 to 34 per fibrinogen.

Suitably, the ratio of unlabeled fibrinogen:labeled fibrinogen ranging from 0:1 to about 50:1, including from about 10:1 to about 50:1.

Suitable labels (also referred to herein as reporters) include a fluorescent label, an absorptive label, a radioactive label, a chromogenic label, and combinations thereof. Suitable fluorescent labels include fluorescein isothiocyanate (FITC), carboxyfluorescein succinimidyl ester (NHS-fluorescein), fluorogenic peptide, chromogenic peptide, and combinations thereof. Suitable radioactive labels include ¹²⁵Iodine (¹²⁵I). Reporter tagging locations and labeling strategy on the clot component can vary, wherein reporters can tag clot components through covalent bonding, which can be achieved through chemical reactions, such as, primary amine conjugations like NHS chemistry, acid chemistry, glycosylation, and disulfide chemistry. Reporters can also label the clot based on size, affinity, and antibody-antigen interactions, for example, quantum dot, reporter tagged peptide, dendrimers and macrocycles, reporter tagged antibody, and combinations thereof. Other component examples like platelets and red blood cells (RBCs) can also be labeled if they are incorporated to form the clot substrate. Reporters that generate intensity based on proximity, distance, or through energy transfer can also be adopted for clot substrate labeling. Subsequent clot digestion monitoring can utilize optical phenomena, for example, fluorescence polarization, fluorescence quenching, fluorescence dequenching, and Förster resonance energy transfer (FRET). The clot substrate can be formed using reporter tagged blood products such as labeled fibrinogen in the presence of clot initiators, wherein blood products can be treated with interventional therapeutics.

In one embodiment, the labeled fibrin clot includes a clot-free detection path.

Proteins and enzymes used in clots or in the sample solution can be of various concentrations and of the same or mixed species of origins. If animal products are used with human products, bovine or porcine origins are preferred human analogs.

The labeled fibrin clot can further include plasma, whole blood, blood serum, synthetic blood, a blood analog, red blood cells, platelets, buffy coat fraction, euglobulin faction, and other known blood components. Suitable plasma includes platelet-rich plasma and platelet-poor plasma. Plasma can be a standardized pooled plasma or patient pathological plasma. Blood products can be independent or a mixture of whole blood, plasma, platelets, RBCs and other blood-related products or components. These products or components can be from or separated from non-anticoagulated fresh blood and/or anticoagulated fresh blood and frozen blood products. Plasma samples can be fresh plasma and anticoagulant treated plasma. The plasma can also be platelet-rich plasma and platelet-poor plasma. Suitable anticoagulants used for blood treatments include sodium citrate, EDTA, heparin and other known anticoagulant agents. As described herein, blood products can be labeled with reporters which can be achieved by, for example, mixing in reporter tagged fibrinogen in ratios. The initiation of the clot can be through recalcification, and/or by the addition of tissue factor, thrombin, phospholipids, or a combination thereof.

The fibrin clot substrate can be modified by components that are added before, during, or after clot formation. These components can modify the clot through covalent or non-covalent bonding/association. For example, they can be coagulation factor XIIIa, von Willebrand factor, plasminogen, tissue plasminogen activators, antiplasmin, crosslinkers like formaldehyde, and a combination thereof. Other suitable components include factors that affect or interrupt the clot formation process, for example, chloride, calcium, and plasmin derivatives. Other suitable components include factors that interact with the completely formed clot based on affinity to cultivate desired clot surface conditions, for example, a synthetic peptide that has both fibrin affinity and plasmin inhibition moiety. Suitable plasminogen activators include tissue plasminogen activator (t-PA), urokinase-type plasminogen activator (u-PA), streptokinase plasminogen activator (strepto-PA), recombinant tissue plasminogen activator (rt-PA), recombinant plasminogen activator (r-PA), tenecteplase (TNKASE®), and combinations thereof. The labeled fibrin clot can include other coagulation factors such as coagulation factor IV, coagulation factor V, coagulation factor VII, coagulation factor IX, coagulation factor X, coagulation factor XI, coagulation factor XII, coagulation factor XIIIa, von Willebrand factor, thrombin activatable fibrinolysis inhibitor (TAFI), and combinations thereof. The labeled fibrin clot can include fibrinolytic factor inhibitors such as TAFI, plasminogen inhibitors (PAI-1), antiplasmin, and combinations thereof.

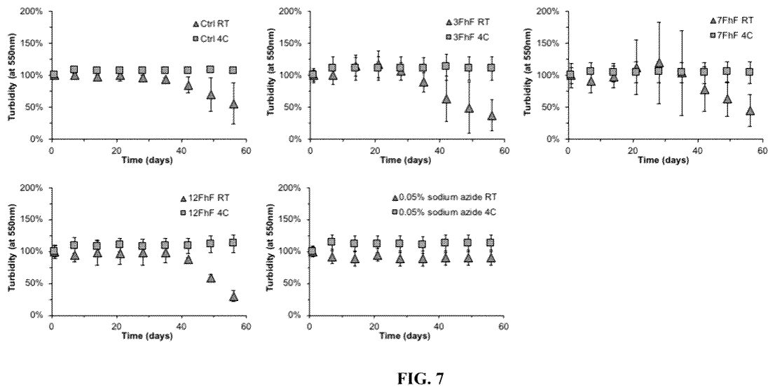

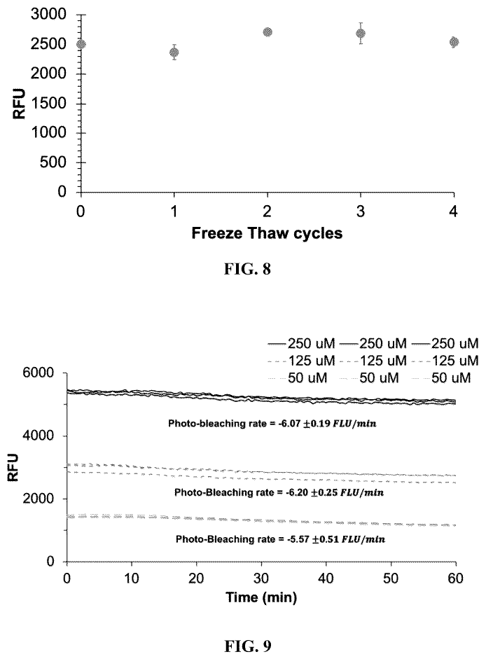

Labeled fibrin clot substrates can be stored for long-term use through refrigeration. Labeled fibrin clots can also include stabilizing agents, for example, sodium azide as briefly illustrated in FIG. 7. Labeled fibrin clots can also be lyophilized and rehydrated prior to use.

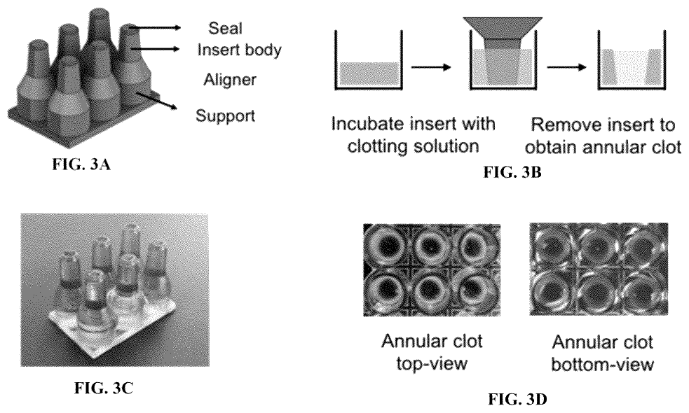

Labeled fibrin clots are accommodated in a container for intensity monitoring by a spectrometer, fluorometer, absorbance meter, or radioactive meter. Suitable containers include single and multi-well plates such as 96-well microplates a representative container as briefly illustrated in FIG. 3. Other suitable container examples include 384-well plates and cuvettes. Labeled fibrin clots can also be adapted for use in a cartridge to be read at point of care or in a clinical core laboratory in an automated fashion.

In another aspect, the present disclosure is directed to a method of analyzing fibrin clot lysis. The method includes: contacting a thrombolytic agent with a labeled fibrin clot, wherein the labeled fibrin clot comprises a ratio of unlabeled fibrinogen:labeled fibrinogen ranging from 0:1 to about 50:1, wherein the labeled fibrinogen comprises from 1 to 34-labels per fibrinogen, and a clot-free detection path; and analyzing the labeled fibrin clot.

The method is particularly suitable for studying thrombosis pathologenesis and thrombolysis.

The thrombolytic agent can include a variety of blood products or buffer solution with or without interventional therapeutics. The thrombolytic agent (alone or including interventional therapeutics) can be added in the center (to the clot-free detection path in the clot). Interventional therapeutics include anticoagulant agents and their inhibitors, for example, heparin, direct oral anticoagulants (DOACs), other novel anticoagulant agents, and synthetic inhibitors; fibrinolytic agents and their inhibitors, for example, plasmin and derivatives, tissue plasminogen activators, thrombin activatable fibrinolysis inhibitor (TAFI), plasminogen inhibitors (PAI-1), plasmin inhibitors; antiplatelet agents and their inhibitors, for example, aspirin. Extra drug delivery vehicles for example, synthetic molecules with drug binding moieties, micelles, dendrimers, lipid vesicles, and combinations thereof, can also be included.

Formation of the labeled fibrin clot and components included in the clot are described herein.

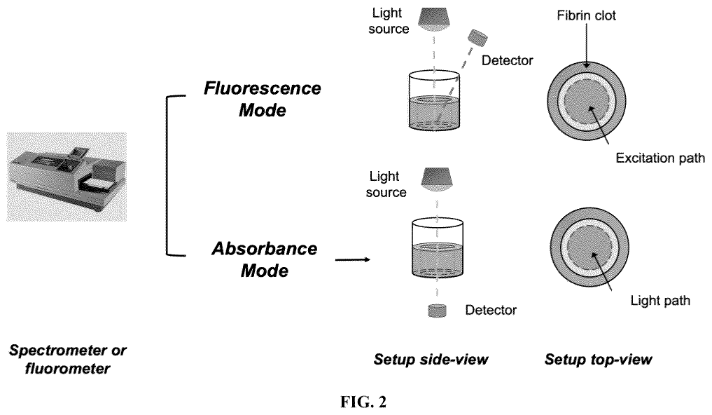

The labeled fibrin clot is suitably analyzed using thromboelastography, turbidity assay, microscopy including fluorescence microscopy and scanning electron microscopy, fluorometry, spectrometry, and combinations thereof. Corresponding reporting techniques can include absorptive, fluorescent (small molecule or quantum dot as representative examples), radioactive, and chromogenic. A common commercially available spectrometer is used to obtain fluorescence and absorbance reads scoping at a certain radial region within the center of each microplate well. Optical measurements can use wavelengths including ultraviolet, visible, near infrared, and combinations thereof. Near infrared is particularly useful for monitoring a sample or labeled fibrin clot substrate component that has auto-fluorescence such as red blood cells. Clot digestion, for example, can be analyzed by monitoring fluorescence intensity as illustrated in FIG. 2. In some embodiments, the fibrin clot substrate can be formed without externally added reporters, for example, ultraviolet absorbance can be used to track fibrin degradation products released from the clot where an unlabeled clot substrate is used. For example, absorbance or fluorescence of hemin can be used to monitor clot digestion when red blood cells (RBCs) are incorporated in the clot substrate.

The method of analyzing fibrin clot lysis can further include analyzing a sample obtained from the clot-free detection path of the labeled fibrin clot. In some embodiments, labeled fibrin, fibrin digestion products, and any other native or non-native protein, peptide, small molecule can be externally analyzed through periodic sampling of the clot free path by a secondary analysis technique such as by histology, ELISA, electrophoresis, or western blot.

In another aspect, the present disclosure is directed to a method of determining a patient's response to a thrombolytic therapy. The method includes: obtaining a plasma sample from the patient; mixing the plasma sample with a labeled fibrinogen clotting solution, the labeled fibrinogen clotting solution comprising: unlabeled fibrinogen and labeled fibrinogen at a ratio of unlabeled fibrinogen:labeled fibrinogen ranging from 0:1 to about 1:0, wherein the labeled fibrinogen comprises from 1 to 34-labels per fibrinogen; forming a labeled fibrin clot by mixing the labeled fibrinogen clotting solution with a clotting activator; forming a clot-free detection path in the labeled fibrin clot; contacting the labeled fibrin clot with a thrombolytic therapy; and analyzing the labeled fibrin clot.

Formation of the labeled fibrin clot and components included in the clot are described herein.

Suitable methods for analyzing the clot and samples obtained from the clot-free detection path are described herein.

Thrombolytic therapy includes a thrombolytic drug candidate. Suitable thrombolytic drug candidates include plasminogen activators. Suitable plasminogen activators include tissue plasminogen activator (t-PA), urokinase-type plasminogen activator (u-PA), streptokinase plasminogen activator (strepto-PA), recombinant tissue plasminogen activator (rt-PA), recombinant plasminogen activator (r-PA), tenecteplase, other novel plasminogen activators, and combinations thereof. Other suitable thrombolytic drug candidates include plasmin, plasmin derivatives, alfimeprase, and combinations thereof.

In another aspect, the present disclosure is directed to a high-throughput screening method for analyzing a candidate for blood clot digestion and thrombolytic agents. The method includes: forming a labeled fibrin clot in a well, wherein the labeled fibrin clot is formed by: labeling fibrinogen to obtain a labeled fibrinogen, wherein labeled-fibrinogen comprises from 1 to 34-labels per fibrinogen; mixing the labeled fibrinogen with an unlabeled fibrinogen at a ratio ranging from about 0:1 to about 1:0 (labeled fibrinogen:unlabeled fibrinogen) to form a labeled fibrinogen clotting solution; contacting the labeled fibrinogen clotting solution with a clotting activator; placing the labeled fibrinogen clotting solution with the clotting activator in the well; performing a clotting step by incubating the labeled fibrinogen clotting solution with the clotting activator in the well for a sufficient time to allow the fibrinogen clotting solution with the clotting activator to form a labeled fibrin clot; and forming a clot-free detection path in the labeled fibrin clot; adding a candidate blood clot digestion or thrombolytic agent to the labeled fibrin clot; and analyzing the labeled fibrin clot.

Formation of the labeled fibrin clot and components included in the clot are described herein.

Suitable methods for analyzing the clot and samples obtained from the clot-free detection path are described herein.

Suitable candidates for blood clot digestion and thrombolytic agents thrombolytic drug candidates as described herein.

In another aspect, the present disclosure is directed to a high throughput screening method for analyzing molecules released from a biological sample. The method includes: culturing the biological sample in a well; forming a cell-free detection path in the biological sample; adding a labeled molecule in the cell-free detection path; adding a candidate molecule; and analyzing the release of molecules.

Analysis includes analyzing a sample obtained from the cell-free detection path, analyzing the biological sample, and combinations thereof.

Suitable methods for analyzing a sample obtained from the cell-free detection path and the biological sample are described herein and include turbidity assay, microscopy, fluorometry, spectrometry, histology, ELISA, western blot and combinations thereof.

Suitable labeled molecules include chromogenic and fluorogenic substrates, and can be based on enzyme-substrate interaction, affinity, immunochemistry and combinations thereof.

Suitable labels include a fluorescent label, an absorptive label, a radioactive label, a chromogenic label, and combinations thereof. Suitable fluorescent labels include fluorescein isothiocyanate (FITC), carboxyfluorescein succinimidyl ester (NHS-fluorescein), fluorogenic peptide, chromogenic peptide, and combinations thereof.

Suitable candidate molecules include agonists and stimulators. Suitably, the candidate molecule(s) are added to the cell-free detection path, the biological tissue, and combinations thereof.

Suitable biological samples include tissues, islets, cells, and cell clusters. In some embodiments, endothelial cells are cultured in the well, chromogenic substrate and agonists such as thrombin can be added in the cell-free detection path to induce PAI-1 release and monitoring.

The method of labeling can further include feeding biological sample with labeled protein factors, DNA or signaling molecules such as calcium.

In some embodiments, fluorophore labeled calcium release from incubated platelet cells can be monitored in the presence of agonists such as ADP and arachidonic acid.

The method of creating a cell-free detection path can include the formation of solid tissue or cell aggregates away from the detection path, coating cells to the surroundings using methods such as extracellular matrix coating, RGD sequence coating or plasma treatment, or using a mechanical measure such as a mesh that have smaller pores than the employed cells.

### Examples

### Materials and Methods

### FITC-Fibrinogen Conjugation

Human fibrinogen and thrombin lyophilized powder (Sigma Aldrich, St. Louis, MO) were reconstituted in phosphate buffer saline (PBS) and deionized water (DI), respectively. FITC (Sigma Aldrich, St. Louis, MO) was reconstituted in PBS with 10% dimethyl sulfoxide (DMSO) to improve solubility. FITC labeled human fibrinogen (FhF) was made by mixing 200-fold excess FITC to fibrinogen at room temperature in PBS for 10 minutes, 120 minutes, and overnight to attain three different FITC per fibrinogen labeling levels (<8% DMSO in reaction solution). Following conjugation, tagged fibrinogen was purified from unreacted FITC using serial dilutions in 100 kDa molecular weight cutoff centrifugal filters following manufacturer recommendations (AMICON®, Millipore, Burlington, MA). FITCs per fibrinogen were determined to be 3-, 7-, and 12-FITC per human fibrinogen comparing 280 and 494 nm absorptivity via a spectrometer (MOLECULAR DEVICE® SpectraMax M5, San Jose, CA), calculated through Beer's law and molar extinction coefficients, which are ε=513, 400 L mol⁻¹ cm⁻¹ (at 280 nm) for fibrinogen and ε=75,855 L mol-1 cm-1 (at 494 nm) for FITC. FITC absorbance contribution at 280 nm was subtracted based on a FITC spectrum when determining fibrinogen concentration. FhFs were aliquoted and stored at −20° C. and freeze-thawed before experiments. These FhF products were experimentally found to be active for clot formation and stable with no change in absorbance (280 nm and 494 nm) or fluorescence intensity for consecutive 4 freeze-thaw cycles (FIG. 8) and over months.

### Clotting Solution Preparation

Unmodified fibrinogen was mixed with 3,7,12-FhF at fibrinogen to FhF ratios of 1:0 (unmodified fibrinogen control), 5:1, 10:1, 30:1, 50:1, and 0:1 (neat FhF). All fibrin clots were formed at a final concentration of 3 mg/mL fibrinogen and 1 U/mL thrombin in PBS.

### FITC-Fibrin Turbidity and TEG Assays

Clot characterization via clot turbidity and TEG assays were described previously. Clot turbidity assays were initiated by mixing thrombin with fibrinogen in a 96-well plate (CORNING®, Corning, NY) and monitored at 550 nm absorbance for 30 minutes via a spectrometer. Turb^{max} (maximum turbidity) was derived from the turbidity tracing curve. TEG assays were initiated by mixing thrombin with fibrinogen in a clear TEG cup and monitored for 30 minutes in a TEG 5000 Analyzer (HAEMONETICS®, Braintree, MA). TEG^{max} (maximum amplitude or MA) was derived by the TEG software (HAEMONETICS®, Braintree, MA).

### Scanning Electron Microscopy

Fibrin clots (80 μL) were fixed by 2.5% glutaraldehyde (Electron Microscopy Sciences Supplier, Hatfield, PA) in PBS solution overnight and washed with DI water five times. Clot samples were then fully dehydrated via overnight lyophilization (FreezeZone 2.5, LABCONCO). It is important to note that the fibrin dehydration process was critical to preserve its micro-morphology under SEM since clots formed in this study were at a concentration of only 0.3% (w/v). Additional dehydration methods were also assessed. Dehydrated samples were further sputter coated with gold (Denton Vacuum Desktop V) for 30 seconds at 3*10⁻⁴ Torr to obtain a ˜10 nm gold coating for SEM. Fibrin micro-structural images were taken using a field emission scanning electron microscopy (JSM-7800F, JEOL) at an acceleration voltage of 5 kV. Fiber diameters, pore size, pore counts, and total pore area were quantified using an open source Image J software.

### Confocal Microscopy

Selected fibrin clots were prepared in a 35 mm glass-bottom dish (MaTek Corporation) microscope dish at a volume of 40 μL. Images were acquired using LSM 800 confocal microscope (Zeiss, Germany) equipped with a C-Apochromat 40×/1.20 W Korr objective. FITC was excited at 488 nm and collected at 519 nm (max emission). Confocal fluorescence images were analyzed using ImageJ.

Fibrin Clot Stability after Storage

FITC-Fibrin clots and unmodified fibrin clots were formed in a 96 well plate at 200-μL and stored at RT and 4° C. for comparison. Longitudinal fibrin clot stability was tested by tracking turbidity at 550 nm over 56 days.

### Annular Clot Fabrication

The annular clot molding insert was designed and drafted using an open source CAD software (Fusion® 360) based on dimensions of an UV transparent 96-well plate (CORNING® 3635) (see, FIG. 3A). The insert was 3D printed on a STRATSYS® Connex 3 printer providing for high precision with a build layer as fine as 16 μm. The body of the insert used an acrylic-based material (VEROCLEAR® RGD810) to ensure a smooth surface (FIG. 3C). The elastomeric end (TANGOPLUS® FLX930) was later cured at the end of the insert to prevent unwanted clot formation in the light path at the base of the well. Annular clots were formed by directly adding clotting solution to the plate at 80 μL and immediately placing the DI water rinsed 3D printed insert into the well (see, FIG. 3B). The insert was gently pressed during the first 2 minutes to ensure a good bottom seal. After 30 minutes clotting at room temperature, the insert was carefully removed and the annular clots were gently washed with 0.01 M PBS twice and stored in 120 μL PBS before use (see, FIG. 3D).

### Dose Response Experiment

Lyophilized plasmin, plasminogen (Athens Biotech, Athens, GA) and tPA (ALTEPLASE® Genentech, San Francisco, CA) were reconstituted in DI water. Sample solutions were made by diluting stock to targeted concentrations with PBS. Plasmin dose-response experiments were conducted at 0.01 to 1.5 U/mL unless otherwise specified. For experiments with varying plasminogen and fixed tPA level (500 ng/ml), 0 to 87.2 μg/mL plasminogen were examined. For experiments with varying tPA and fixed plasminogen level (58.1 μg/mL), 0 to 1000 ng/ml tPA were examined.

### Annular Clot Lysis Assay

In all experiments, 120 μL sample solutions were added to the center of the annular clot to initiate clot lysis. Fluorescence (Ext 495, Em 519) was monitored for 60 minutes at a 30 second interval. All clot lysis experiments were performed at 37° C. in triplicate. The Limit of Detection (LoD) is defined as the lowest analyte concentration reliably distinguished from the blank. Thus, LoDs of annular clot lysis assay were determined based on the measured Limit of Blank (LoB) and the lowest concentration of plasmin that has a significantly different fluorescence release rate compared to the blank. Analytical sensitivity was determined by the slope of a linear fitted line at lower plasmin concentrations (<0.05 U/mL).

### S2251 Chromogenic Assay

S2251 assays were performed in non-binding 96 well plates (CORNING® 3641). Sample solutions were pipetted into the well containing 500 μM S2251 chromogenic substrate (Diapharma, West Chester, OH) at a final volume of 100 μL. p-Nitroaniline (pNA) absorbance (405 nm) was monitored for 10 minutes at a 30 second interval. All experiments were performed at 37° C. with experimental groups in triplicates. V₀ (Initial velocity) was derived using the initial linear region of the absorbance tracing curve.

### Statistical Analysis

All results were reported as mean±standard deviation. Two tailed student t tests were performed and significant statistical differences were reported at P values <0.05. P values lower than 0.01 and 0.001 were also indicated as appropriate in plots and tables. Linear regressions were fitted in some plots and corresponding r square values were reported. The detailed protocols were specified in corresponding figure legends.

### Results

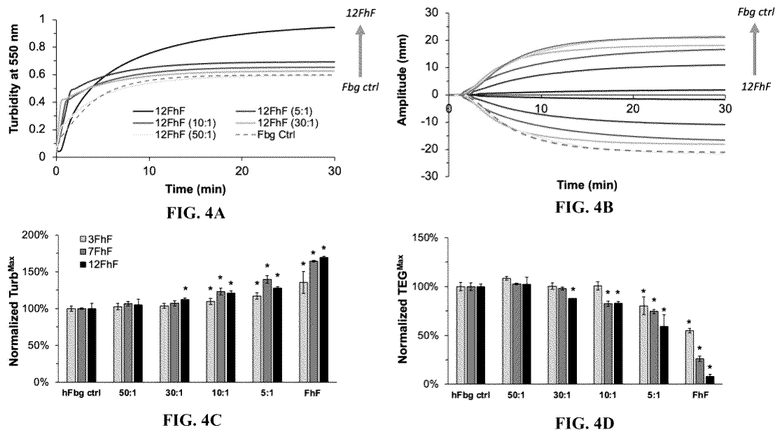

The clinical relevance of an in vitro fibrin clot lysis assay relies on a representative clot substrate that has non-disrupted physical and biological properties. Since the FITC-fibrinogen conjugation is mediated through the covalent bond, it is necessary to examine the fibrinogen tagging effect on clot properties. In this Example, FITC labeled human fibrinogen (FhF) were made by incubating human fibrinogen with FITC at a 200 fold excess. Although a single fibrinogen molecule has around 200 lysine residues, a maximum of 13 FITC per fibrinogen was obtained. By increasing the incubation time from 10 minutes, 2 hours to overnight, 3-, 7-, and 12-FITC per fibrinogen were fabricated as determined via spectrometer using 494 nm to 280 nm absorbance conversion derived from the FITC absorbance spectrum. To assess the effects of fibrinogen tagging on fibrin clot formation, bulk clot strength and fibrin fiber packing were examined by TEG and turbidity assays. Turbidity and amplitude were tracked over time for clots formed at increasing levels of FITC-fibrinogen (FIGS. 3A and 3B). Turb^{Max} and TEG^{Max} were obtained for analysis and data were normalized to that of unmodified human fibrinogen to eliminate batch-to-batch variation (9% for Turb^{Max} and 4% for TEG^{Max}) and allow for direct comparisons of percentage change across samples. All tested sample turbidities were within the detection limit of the spectrometer and were baseline-subtracted before data normalization. FITC absorbance at 550 nm at concentrations used in this Example was negligible. As was expected, FITC-fibrin(ogen) conjugation rendered a significant impact to both clot strength and macroscopic clot structure as determined by turbidity. Overall, increasing FhF levels (FhF to fibrinogen ratio) in the clot contributed to higher Turb^{Max} (maximum turbidity) and lower TEG^{Max} (clot strength) which was consistently observed in all three groups (3-, 7-, and 12-FhF) (FIGS. 4A and 4B).

Specifically, neat 3-, 7-, and 12-FhF clot samples showed 45, 74 and 92% reduction in clot strength and 35, 65, and 69% increase in clot turbidity compared to unmodified fibrinogen, respectively. 12-FhF showed very low TEG^{Max} and high Turb^{Max} amidst all neat samples indicating a larger disruption of fibrin polymerization due to an increase of FITC-conjugations per fibrinogen. At lower FhF ratios, clot strength and clot turbidity values leveled off and matched values of the unmodified fibrinogen sample. The inverse tracking of clot strength and clot turbidity at increased FhF ratios in fibrin resembled what was observed in experiments on varying multiple other clotting variables. The combination of high turbidity and low clot strength at a constant fibrinogen level demonstrated formation of thick fibers and a loose fibrous fibrin network. Time to maximum clot formation was also determined to assess the impact of fibrinogen tagging on the process of clotting. In both assays, most samples showed resembling time to maximum clot formation with a difference less than 20% indicating similar clotting performance across groups. However, neat 12-FhF sample showed three times longer time to maximum clotting compared to all other samples due to its slow clotting progression and low ultimate clot strength.

The physiologically relevant fibrin clotting mixture was determined through matching clot properties of a sample to that of the unmodified fibrin clot. The 7-FhF (30:1) and 12-FhF (50:1) groups were determined to be physiologically relevant as their Turb^{Max} and TEG^{Max} showed no statistical difference (P<0.05) above 30:1 and 50:1, respectively. 3-FhF (10:1) was also selected as a physiologically relevant mixture as TEG^{Max} showed no statistical difference while Turb^{Max} showed less than 10% difference.

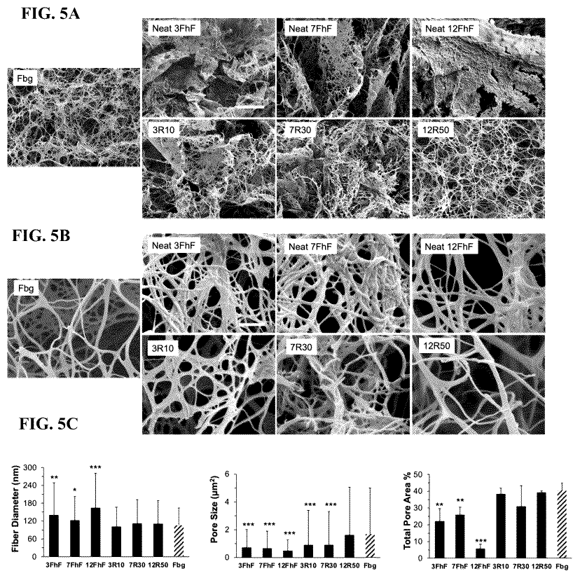

To directly assess clot morphology at varying ratios of tagged and untagged fibrinogen, an array of fibrin clot mixtures was analyzed by SEM. These clots included neat FhF, unmodified fibrin clots, and three physiologically relevant FhF clot samples-3-FhF (10:1), 7-FhF (30:1) and 12-FhF (50:1). Clots were formed, crosslinked, lyophilized and gold coated. Clot microstructures were compared at 4,000× and 15,000× magnifications (FIGS. 5A and 5B).

Neat FhF clot samples exhibited scale-like patterns and fused fibrin fiber morphology. These clots were further categorized as having larger fiber diameter, smaller pore size and smaller total pore percentage area compare to other groups (FIG. 5C). A quantitative analysis was conducted using Image J. Pore size and total pore area were measured using a thresholding method where the same threshold level was applied across all the samples throughout the analysis. Based on the quantitative results, the neat 12-FhF clot sample showed the most unique and dissimilar SEM morphology, i.e. thickest fiber and lowest pore area. These direct morphological measurements were consistent with the bulk clot structure as assessed by clot turbidity. The physiologically relevant clot groups showed much cleaner fibrin morphology that was more similar to that of neat FhF clots.

Despite having similar TEG and turbidity values as unmodified fibrinogen, not all physiologically relevant groups exhibited a native fibrin structure under SEM. 3-FhF (10:1) and 7-FhF (30:1) clots showed similar fiber diameters compared to unmodified fibrin clots, but both groups had significantly smaller pore size and a moderate level of fused fibrin fiber morphology. Only the 12-FhF (50:1) sample showed minimal differences, both qualitatively and through defined characteristics, compared to the unmodified fibrin control. This indicated that the ratio of fibrinogen to FhF rather than the amount of FITC per fibrinogen exerted a larger ultimate impact on fibrin structure upon clotting. To examine this, the effect of fibrinogen to FhF ratio on clot micro-morphology was analyzed under SEM. In this experiment, clots were formed by decreasing 12-FhF ratios (fibrinogen to FhF=0:1, 10:1, 30:1, 50:1, 1:0) in the fibrin. Results showed that the presence of more FhF contributed to clots with significantly larger fiber diameters, reduced pore size and total pore area. This confirmed that increasing FITC-fibrin(ogen) levels in fibrin clots disrupted clot properties resulting in less physiologically relevant fibrin clots. In summary, the clotting solution of unmodified fibrinogen with 12 FITC per human fibrinogen at 50:1 ratio produced the most physiologically relevant fibrin clot as determined by TEG, Turbidity, and SEM.

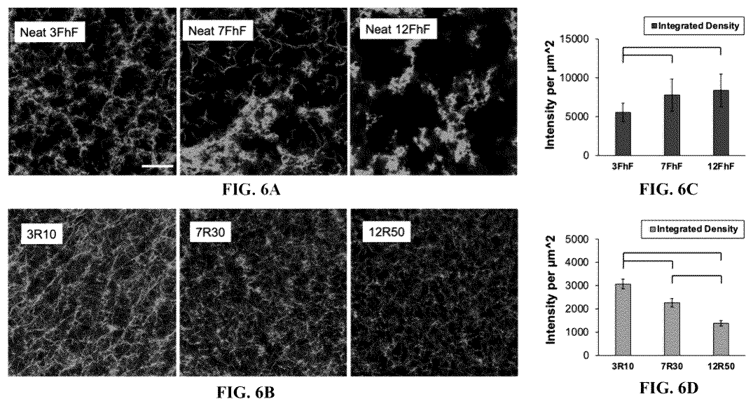

To assess FITC labeling homogeneity, confocal microscopy images of neat and physiologically relevant 3-, 7-, 12-FhF formed fibrin samples were taken at 3 different locations with 5 different spots at an interval of 10 μm in the vertical direction. Three images at locations 30 μm below the surface were used for comparison and quantitative analysis. Neat FhF clot samples exhibited bright images with significant fluorescent aggregates at higher FITC per fibrinogen whereas the three physiologically relevant FhF clots showed homogeneous FITC labeling throughout their fibrin structures (FIGS. 6A and 6C). Unmodified fibrin clots could not be imaged utilizing this technique as they did not exhibit endogenous fluorescence in the absence of FITC tagging.

Neat and physiologically relevant (PR) FhF clot samples were excited at 0.2% and 1% energy levels to avoid signal underexposure or saturation, respectively. Images were taken immediately after excitation to avoid photobleaching over time. Integrated intensities were reported in fluorescence unit (FLU, or arbitrary units) per μm² by averaging multiple images at different depths for each sample in bar plots (FIGS. 6B and 6D). Neat FhF clots showed higher integrated intensity at increased FITC per fibrinogen. However, the value of the neat 12-FhF sample was only 1.5 times that of the neat 3FhF sample while the FITC concentration ratio of these two samples was 4 times, representing a reduction in intensity per FITC of 62%. This large reduction was indicative of local fluorescence quenching due to increasing FITC proximity on fibrinogen which has been reported. Comparing 12-FhF (50:1) to 3-FhF (10:1) samples, integrated intensity ratios and FITC concentration ratios were also mismatched contributing to a 64% reduction of integrated intensity per FITC in the former group. 7-FhF neat and 7-FhF (30:1) samples also showed 39.6% and 42.7% reductions in integrated intensity per FITC compared to 3-FhF neat and physiologically relevant samples, respectively. Since similar levels of reduction in integrated intensity per FITC were observed for neat and physiological relevant samples, fluorescence quenching was dominated by intra-fibrinogen rather than inter-fibrinogen when they were incorporated in the fibrin clot. This quenching effect was largely due to the use of FITC as a fluorescent probe and can be mitigated by selecting a probe that is less prone to self-quenching.

The labeling homogeneity was quantitatively assessed by the intensity fluctuation percentage, which was derived by dividing the average integrated intensity in a total of five images by standard deviation. All neat FhF samples showed >20% fluctuation in fluorescence intensity with obvious fluorescence aggregation while physiologically relevant FhF samples exhibited less than 10% fluctuation. This confirmed that physiologically relevant FhF samples had an overall better labeling homogeneity across the clot. In addition, unlabeled area among fluorescently labeled fibers were also quantified using thresholding method in ImageJ. The unlabeled area was a function of both empty space in the clot, and fluorescent labeling density. While the physiologically relevant clot samples shared similar clot structure under SEM, the 3-FhF (10:1) showed 63.0%+1.1% unlabeled area which was significantly lower than 76.2%+6.7% (P<0.001) for 7-FhF (30:1) and 80.9%+7.2% (P<0.001) for 12-FhF (50:1). This result was expected due to 3-FhF physiologically relevant FhF clot sample having the highest labeling ratio of 10:1 among the three groups. Based on clot characterization results from clot turbidity, TEG, and SEM, 12-FhF (50:1) showed the best match to unmodified fibrin. Although its fluorescence labeling may not be the best in terms of signal intensity via confocal microscopy among all three physiological relevant formula, the 12-FhF (50:1) clotting mixture still showed good labeling homogeneity which potentiates an uniform tracking of fluorescence signal during clot lysis.

Fluorescently labeled fibrin digestion cannot be monitored directly through the labeled substrate to track clot digestion due to the saturating level of fluorescence tag present in the excitation path. A common solution is to allow digestion for a period of time before removing digestion supernatant for a reading. However, this procedure prevents a real time clot digestion tracking. The annular clot geometry of the present disclosure provides a unique solution to the simplification of fluorescently labeled clot lysis monitoring. This setup not only enables a real-time signal tracking and increases assay-multiplexing potential but also largely eliminates experimental artifacts. As determined from the characterization experiments in previous section, the 12-FhF (fibrinogen to FhF=50:1) was selected as the clotting mixture to form the physiologically relevant FITC-fibrin. The annular clots were made by placing an insert into the clotting mixture right after a clot initiation through mixing thrombin with neat or physiologically relevant 12-FhF solution. The molding insert removal process was smooth with no repellence or visible interruption to the clot surface. The annular clot showed an average volume of 60.5±0.8 μL and an averaged background value of 798±319 fluorescence unit (n=114). With the addition of PBS solution into the clot, no degradation of fibrin was observed for the length of experiment by monitoring absorbance at 280 nm. Overall, the described annular clot method was successful and highly reproducible.

Plasmin is the protease responsible for fibrinolysis in the plasma. Its activity directly contributes to the fibrinolytic potential of a plasma sample. Maximal inducible plasmin activity in plasma is about 1 U/mL following full activation of endogenous plasminogen and exhaustion of plasmin inhibitors. To examine the fibrinogen tagging effect on fluorescently labeled fibrin clot digestion, increasing amounts of plasmin were tested comparing S2251 chromogenic substrate to neat 12-FhF and 12-FhF (50:1) annular clots. In the S2251 assay, the plasmin dose-response result was reported by plotting the initial velocity V₀ (Abs/min) over plasmin concentrations. A linear relationship was observed between V₀ and plasmin concentration. At plasmin levels above 1 U/mL the substrate digestion rate occurred too rapidly to obtain an accurate initial velocity by the spectrometer. In all, the S2251 assay showed good linearity with increasing plasmin levels from 0.01 to 1 U/mL.

Unlike digesting evenly mixed, small soluble peptide substrate in the S2251 assay, plasmin digestion of thick and insoluble fibrin such as the present annular clot involved solid-liquid phase interactions and molecule transport. Initial velocity measurement that worked for chromogenic assays was not applicable to the annular fibrin clot lysis assay. In this assay, fluorescence signal was smoothly tracked over time at a 30 second interval exhibiting an overall faster fluorescence release at a higher plasmin concentration. Contrary to a linear signal tracing monitored for S2251 digestion, annular clot lysis showed a lag phase followed by a fast digestion phase and an ultimate plateau phase (FIG. 10A). The lag phase can be explained by the cause of plasmin diffusion due to the static nature of the assay. The diffusion of plasmin was not solely based on its relative hydrodynamic size to fibrin pores but rather dependent on plasmin-fibrin bindings. Recent in vitro experiments have confirmed that diffusion of plasmin(ogen) is restricted within a thin fibrin layer (5-8 μm) due to fibrin bindings. A potential co-contributor to the lag phase is the gradual exposure of more binding sites, i.e. C-terminal lysine residues, by plasmin accumulation to the digestion front. The exposing rate can be affected by protease concentration and the presence of protease inhibitors. In the annular clot lysis assay, the lag phase is useful to examine the initial interactions of the protease and fibrin. To conduct a quantitative assessment, FLU200 was used which defines the time takes to reach a 200 fluorescence unit. At increasing plasmin levels, FLU200 time showed a decreasing trend (FIG. 10B). In the fast digestion stage where fibrin binding moieties are relatively abundant, digestion rate better reflected the maximal fibrinolytic activity of the sample. Therefore, VFR (fluorescence release rate, FLU/min) was derived by taking the maximum velocity (10 min period) to represent clot digestion rate in this assay. At lower plasmin concentrations (<1 U/mL plasmin), although lag and fast digestion phases can hardly be distinguished since C terminal lysine residuals are constantly larger than plasmin amount, digestion rates were still determined at the fastest 10-min linear period.

Neat and physiologically relevant 12FhF mixtures were compared in the annular clot lysis assay to determine the fibrinogen tagging effects on fibrinolysis. Overall, both annular clot lysis assays showed that VFR increased and leveled off at higher plasmin concentrations, which agreed with clot digestion at increased plasmin monitored by the fibrin plate method. To derive simple calibration equations to determine plasmin activity in unknown samples, the VFR and plasmin concentrations were plotted in a double-logarithmic scale (FIG. 11A). Data points of both groups showed good linear regressions with r square values >0.95. Neat and physiologically relevant 12 FhF groups shared similar slopes indicating a comparable dose-response performance across tested plasmin concentrations. Whereas, the analytical sensitivity of the neat 12 FhF substrate was almost 20 times higher than its physiologically relevant counterpart (FIG. 11B). Its LoD was also slightly lower giving a 1.5 mU/mL comparing to 6.9 mU/mL for the physiologically relevant 12FhF clot. The digestion performance of both substrates was further compared using equivalent fibrin degradation rates (VFDR). VFDR of physiologically relevant and neat 12FhF were converted via multiplying their VFR values by 50 and 1, respectively. It was anticipated that neat FhF clots would have larger VFDR at these plasmin concentrations since fibrin composed of thick and loose fibers usually exhibit faster clot digestion. Unexpectedly, VFDR of physiologically relevant 12 FhF clots were 16 to 22 times faster than those of neat 12 FhF clots at tested plasmin concentrations. It can be concluded that increased FhF levels impair fibrinolysis to a large extent. In all, despite the higher levels of signal associated with the neat 12FhF, its digestion was considerably slower than the PR-12FhF clots. This reduced digestion rate can be attributed to the tagged fibrin impairing plasmin's ability for fibrin digestion. For applications in which the highest clot digestion signal is desired, the neat 12FhF can be utilized. For applications using a physiologically relevant clot substrate, preparing a 12FhF (50:1) mixture can be utilized.

In plasma, tPA cleaves plasminogen into plasmin that initiates fibrinolysis. The catalytic efficiency of plasminogen activation by tPA has been reported to be orders of magnitude higher in the presence of fibrin than it is in the absence. The S2251 assay has commonly been used to assess urokinase or streptokinase initiated plasminogen activity lacking the ability to examine plasminogen activation by tPA due to the absence of fibrin. To demonstrate the benefit of using annular clot lysis assay over this chromogenic substrate, digestion solutions made by combining tPA and plasminogen were tested in the physiologically relevant 12FhF annular clot and S2251 assay. Varying plasminogen at fixed tPA and varying tPA at fixed plasminogen dose-response experiments were performed. V₀ (in S2251) and VFR (in annular clot lysis assay) over components concentration were plotted using the primary axis. Equivalent plasmin activities were computed using equations derived from plasmin dose-response plots and were reported in U/mL in plots on the secondary axis (FIGS. 17A, 17B, 17D and 17E). At increased plasminogen levels and a fixed 500 ng/mL tPA, S2251 assay reached its detection limit at ˜29 μg/mL plasminogen. Conversely, VFR from the annular clot lysis assay showed a clear increasing trend until a level-off at concentrations above 58.1 μg/mL.

At increased tPA levels and a fixed 58.1 μg/mL plasminogen, both V₀ and VFR increased and showed a tendency to level off at higher tPA concentrations. Plasmin activities were compared across and within assays. In the absence of fibrin, the S2251 assay showed extremely low plasmin activity values converted using the standard curve and the equation derived in the plasmin dose-response experiment. Annular clot lysis assay showed a two orders of magnitude larger (P<0.05) plasmin activity values than those of S2251 assay at all tested groups for both varying tPA and varying plasminogen experiments. Within annular clot lysis assay, plasmin activities showed significant differences across all groups (P<0.05) when varying tPA. In experiment varying plasminogen, all groups showed significantly different plasmin activities compared to 2.9 μg/mL (P<0.05) and all groups except 29.1 μg/mL showed significant differences compared to 5.8 μg/mL (P<0.05). These results confirmed that the annular clot lysis assay was capable of differentiating digestion rate or plasmin activities at a wide range of plasminogen or tPA levels. Overall, FLU200 decreased and leveled off at higher plasminogen or tPA levels (FIGS. 6C and 6F). Importantly, the FLU200 of groups in these experiments were almost 3 times longer than groups with an equivalent VFR in the plasmin dose-response experiment (P<0.05). The extended lag phases in these samples were expected since the activation of plasminogen by tPA and the molecular interaction involving fibrin binding can contribute to elongated preparing time before digestion. These findings also demonstrated the capability of the annular clot lysis assay at picking up interplays of fibrinolytic factors.

The tPA activation of plasminogen with S2251 was also run in the presence of soluble fibrinogen and a similarly low activation of plasminogen was observed. In general, tPA and plasmin(ogen) are key enzymes in the fibrinolytic pathway. Being capable of differentiating tPA and plasminogen levels in the sample, the annular clot lysis assay undoubtedly depicted a more versatile assessment of sample fibrinolytic potential when compared to the S2251 assay. In addition, since tPA or alteplase is commonly used in the thrombolytic therapy for treating acute thrombosis events, the annular clot lysis assay can be conducted as a clinical pilot test to predict patient response to the thrombolytic therapy. With a proper modification of the fibrin clot, for example, by including a patient's own plasma protein, red blood cells and platelets, the annular clot lysis assay can be used to determine a dosing strategy.

The FITC-labeled annular clot lysis assay of the present disclosure provides a convenient method for a reliable assessment of sample fibrinolytic activity. The assay offers a real-time tracking of clot digestion where both a lag phase and a clot digestion rate can be identified and quantitatively compared. Based on these metrics, the Example demonstrated the assay's capabilities of differentiating multiple fibrinolytic factors at physiological concentrations. In addition, the tagged clot substrate can be stored at 4° C., which has been experimentally found to have a long-lasting stability. This greatly expands the utility of the annular clot lysis assay of the present disclosure, especially under the fast-responding clinical settings as it does not need to be formed directly prior to use.

The annular clot lysis assay approaches a representative fibrinolytic process by utilizing a physiological relevant fibrin substrate with a concentration gradient-driven sample digestion. FITC labeling in the fibrin clot with a unique annular shape in the 96 well plate facilitates an easy-to-multiplex setup to acquire fibrinolytic information of samples via a spectrometer (or fluorometer). By analyzing the fibrinogen:FhF ratio in the clotting mixture, the impact of FITC to fibrinogen conjugation on bulk fibrin properties such as clot strength and clot turbidity were minimized. Results demonstrated that the fibrinogen to FITC-fibrinogen ratios that mitigate deleterious effects associated with FITC tagging were at 30:1 for 3FhF, 30:1 for 7FhF, and 50:1 for 12FhF. SEM imaging results validated the similarity between 12FhF (50:1) and unmodified fibrinogen. Fibrinolytic activities of solutions containing different levels of plasmin were tested, both neat and physiologically relevant 12 FhF annular clot showed good limit of detection while neat 12FhF annular clot showed 19 times better sensitivity than its physiologically relevant counterpart. Fibrinolytic activities of solutions containing plasminogen and tPA were further examined, the physiological relevant 12 FhF annular clot lysis assay was capable of differentiating digestion at varying fibrinolytic components levels or varying fibrin-binding affinity. In addition, the physiological relevant clotting formula exhibit clots with moderate properties which makes it feasible to tune FITC labeled fibrin clot structure by changing clotting conditions. The clotting conditions can be adjusted to match some pathological conditions while a neat FhF clot usually cannot due to their extreme clot properties. The tunable fibrin clot substrate itself or as a base of a tunable synthetic blood clot can be used to mimic clinical clot or thrombi structures to provide insights into the treatment for thrombosis at specific patient conditions. For instance, annular clots can be made at varying fibrinogen levels to help predict therapeutic dosage for patient with fibrinogen deficiency or hyper-fibrinogen levels as were seen in COVID-19 patients. The adjusted FITC labeling fibrin(ogen) formula can also be introduced to studies that monitor FITC-fibrin digestion under a confocal microscopy because of its modest intensity and labeling homogeneity. Thrombolytic drug efficacy are usually examined in a 125I-fibrinogen contained plasma clot. The annular clot assay advantageously provides an alternative to the 125I-fibrinogen type of study as FITC is a more accessible and less hazardous reporter compared to isotopic iodine.

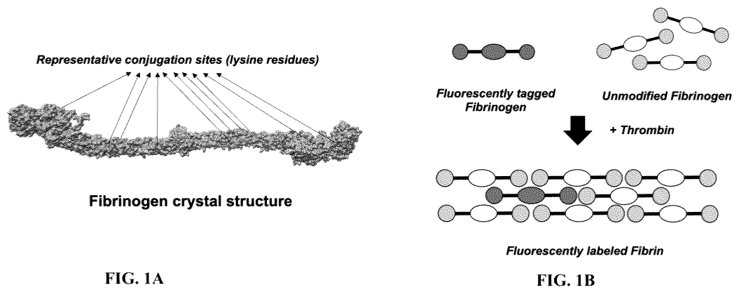

The results provided in the Examples demonstrate a highly reproducible in vitro clot lysis assay that offers a physiologically relevant assessment of sample fibrinolytic activity through a controlled fibrin substrate and easily-multiplexed setup. The assay utilized a FITC-labeled fibrin-based clot forming at physiological fibrinogen and thrombin concentrations. The clot substrate was engineered to be an annular shape and pre-formed in a 96 well plate with the help of a 3D-printed molding insert. Specifically, the unique clot geometry provides for a clear light path for fluorescence excitation and emission by taking advantage of the default signal acquisition mechanism of a commercial spectrometer (FIG. 1). With the addition of fibrinolytic sample solution to digest the annular clot, increasing amount of soluble fluorescently tagged fibrin degradation products are released over time. These fragments disperse into the solution and fluorescence signal is monitored by the detector. This setup enables a real-time measurement of clot lysis with no disturbance during the reading process. To reduce FITC-fibrin(ogen) conjugation impact and establish a physiologically relevant FITC labeled clot, clotting mixture using different FITC per fibrinogen and different FITC-fibrinogen to unmodified fibrinogen ratios were explored. Multiple tools were used to assess fibrin clot characteristics. Clot formation, bulk structure and viscoelastic property were examined by clot turbidity and thromboelastography (TEG) assays. Clot microstructure including fiber thickness and pore size was examined via scanning electron microscopy (SEM). Fluorescence labeling homogeneity and signal density were compared under the confocal microscope. This information was combined to guide FITC labeling in fibrin to achieve a physiologically relevant FITC labeled fibrin clot that is structurally indistinct from an unmodified in vitro statically formed fibrin. The FITC-fibrin clots were further tested for fibrinolysis using samples that contains plasmin or a mixture of exogenous tPA and plasminogen to demonstrate the assay's capacity of differentiating sample fibrinolytic potential or examining drug dose-response.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A labeled fibrin clot comprising: unlabeled fibrinogen and labeled fibrinogen at a ratio of unlabeled fibrinogen:labeled fibrinogen ranging from 0:1 to about 1:0, wherein the labeled fibrinogen comprises from 1 to 34-labels per fibrinogen; and a preformed clot-free detection path in the labeled fibrin clot.

2. The labeled fibrin clot of claim 1, wherein the labeled fibrinogen comprises from 1 to 16-labels per fibrinogen.

3. The labeled fibrin clot of claim 1, wherein the ratio of unlabeled fibrinogen:labeled fibrinogen ranges from 0:1 to about 50:1.

4. The labeled fibrin clot of claim 1, wherein the ratio of unlabeled fibrinogen:labeled fibrinogen ranges from about 10:1 to about 50:1.

5. The labeled fibrin clot of claim 1, wherein the label comprises a fluorescent label, an absorptive label, a radioactive label, a chromogenic label, and combinations thereof.

6. (canceled)

7. The labeled fibrin clot of claim 1, further comprising at least one of plasma, whole blood, synthetic blood, a blood analog, red blood cells, and platelets.

8. (canceled)

9. A method of analyzing fibrin clot lysis, the method comprising:
contacting a thrombolytic agent with a labeled fibrin clot, wherein the labeled fibrin clot comprises
a ratio of unlabeled fibrinogen:labeled fibrinogen ranging from 0:1 to about 50:1, wherein the labeled fibrinogen comprises from 1 to 34-labels per fibrinogen, and a preformed clot-free detection path; and
analyzing the labeled fibrin clot.

10. The method of claim 9, further comprising
preparing a labeled fibrinogen clotting solution;
contacting the labeled fibrinogen clotting solution with a clotting activator;
placing the labeled fibrinogen clotting solution with the clotting activator in a well;
performing a clotting step by incubating the labeled fibrinogen clotting solution with the clotting activator in the well for a sufficient time to allow the fibrinogen clotting solution with the clotting activator to form a labeled fibrin clot; and
forming the clot-free detection path either during the clotting step or after the labeled fibrin clot is formed.

11. The method of claim 9, wherein the label comprises a fluorescent label, an absorptive label, a radioactive label, a chromogenic label, and combinations thereof.

12. (canceled)

13. The method of claim 9, wherein the labeled fibrin clot further comprises plasma, whole blood, synthetic blood, a blood analog, red blood cells, platelets and combinations thereof.

14. The method of claim 13, wherein the plasma, whole blood, red blood cells, and platelets are obtained from a patient.

15. The method of claim 13, wherein the plasma and whole blood further comprises an anticoagulant.

16. (canceled)

17. The method of claim 13, wherein the plasma comprises platelet-rich plasma and platelet-poor plasma.

18. The method of claim 9, wherein the labeled fibrin clot further comprises a plasminogen activator.

19. (canceled)

20. The method of claim 9, wherein the labeled fibrin clot further comprises an anticoagulant.

21. (canceled)

22. The method of claim 9, wherein the labeled fibrin clot further comprises a coagulation factor.

23. (canceled)

24. The method of claim 9, further comprising adding plasminogen.

25. The method of claim 9, further comprising adding a fibrinolytic factor inhibitor.

26. (canceled)

27. The method of claim 9, wherein the thrombolytic agent comprises a thrombolytic drug candidate.

28. (canceled)

29. (canceled)

30. The method of claim 9, further comprising analyzing a sample obtained from the clot-free detection path of the labeled fibrin clot.

31-85. (canceled)
